(19) United States
(12) Patent Application Publication
(10) Pub. No.: US 2015/0059012 A1
(43) Pub. Date: Feb. 26, 2015

(54) PEPPER VARIETY NUN 70030 PPH

(71) Applicant: Nunhems B.V., AC Nunhem (NL)

(72) Inventor: Jesus SALDIVAR, Leon (MX)

(21) Appl. No.: 14/533,350

(22) Filed: Nov. 5, 2014

(51) Int. Cl. A01H 5/08; A01H 1/02; A01G 1/00; C12N 5/04

(52) U.S. Cl. CPC A01H 5/08; C12N 5/04; A01H 1/02; A01G 1/001
USPC 800/260; 800/317.1; 435/410; 800/303; 800/302; 800/301; 800/300; 47/58.1 FV; 426/615

(57) ABSTRACT

The invention relates to the field of Capsicum spp., in particular to a new Capsicum variety designated NUN 70030 PPH plants, seeds and pepper fruits thereof.

### FIELD OF THE INVENTION

The present invention relates to the field of plant breeding and, more specifically, to the development of pepper variety NUN 70030 PPH, also referred to as “NUN 70030” or Emperador.

The goal of vegetable breeding is to combine various desirable traits in a single variety/hybrid. Such desirable traits may include greater yield, resistance to insects or pests, tolerance to heat and drought, better agronomic quality, higher nutritional value, growth rate and fruit properties.

Breeding techniques take advantage of a plant's method of pollination. There are two general methods of pollination: a plant self-pollinates if pollen from one flower is transferred to the same or another flower of the same plant or plant variety. A plant cross-pollinates if pollen comes to it from a flower of a different plant variety.

Plants that have been self-pollinated and selected for type over many generations become homozygous at almost all gene loci and produce a uniform population of true breeding progeny, a homozygous plant. A cross between two such homozygous plants of different varieties produces a uniform population of hybrid plants that are heterozygous for many gene loci. Conversely, a cross of two plants each heterozygous at a number of loci produces a population of hybrid plants that differ genetically and are not uniform. The resulting non-uniformity makes performance unpredictable.

The development of uniform varieties requires the development of homozygous inbred plants, the crossing of these inbred plants, and the evaluation of the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines are developed by selfing and selection of desired phenotypes. The new lines are evaluated to determine which of those have commercial potential.

One crop species which has been subject to such breeding programs and is of particular value is the pepper. Pepper (Capsicum spp.) is naturally a diploid and the basic chromosome number of the genus is x=12, most are 2n=2x=24, including the cultivated ones. A few wild species have 2n=26. Ploidy changes (both tetraploidy and haploidy) are relatively easy to induce in Capsicum species. Doubled haploids have proved particularly valuable in the analysis of the genetically complex basis of some resistances to pests and diseases.

The genus Capsicum originated in American tropics. The fruit of most species of Capsicum produce a strong burning sensation (pungency or spiciness) in the mouth of the unaccustomed eater due to the presence of capsaicin (methyl vanillyl nonenamide), a lipophilic chemical. Capsaicin can be present in large quantities in the placental tissue (which holds the seeds), the internal membranes, and to a lesser extent, the other fleshy parts of the fruits of plants in this genus. The seeds themselves do not produce any capsaicin. The amount of capsaicin in the fruit is highly variable and dependent on genetics and environment, giving almost all types of Capsicum varied amounts of perceived heat. The most recognizable Capsicum without capsaicin is the bell pepper, a cultivar of Capsicum annuum, which has a zero rating on the Scoville scale. The lack of capsaicin in bell peppers is due to a recessive gene that eliminates capsaicin and, consequently, the “hot” taste usually associated with the rest of the Capsicum family.

Many of the peppers currently used which are used in the fresh of processed marked in the United States are seeded hybrid varieties. Hybrid varieties offer the advantages of easy combination of dominant and recessive traits, such as disease resistance, from a set of inbred parents, as well as careful control of parentage.

Many different pepper cultivars have been produced, and pepper breeding efforts have been underway in many parts of the world. Some breeding objectives include varying the color, texture and flavor of the fruit. Other objectives include disease or pest resistance, optimizing flesh thickness, yield, suitability to various climatic circumstances, heat, solid content (% dry matter), and sugar content.

Advances in biotechnology have also resulted in genetically engineered pepper plants with improved traits. For example, fungal resistant pepper plants comprising a PepEST or PepDef gene where the expression of the nucleic acid sequence in the plant resulted in increased resistance to fungal infection (see e.g., US application 20060037100, Feb. 16, 2006).

While breeding efforts to date have provided a number of useful pepper varieties with beneficial traits, there remains a great need in the art for new varieties with further improved traits. Such plants would benefit farmers and consumers alike by improving crop yields and/or quality.

### SUMMARY OF THE INVENTION

In one aspect of the invention, a seed of pepper variety NUN 70030 PPH is provided, wherein a representative sample of said seed has been deposited under Accession Number NCIMB ______.

In another aspect the invention provides for a hybrid variety of Capsicum annuum called NUN 70030 PPH. The invention also provides for a plurality of seeds of the new variety, plants produced from growing the seeds of the new variety NUN 70030 PPH, and progeny of any of these. Especially, progeny retaining one or more (or all) of the “distinguishing characteristics” or one or more (or all) of the “essential morphological and physiological characteristics” or essentially all physiological and morphological characteristics of NUN 70030 PPH referred to herein, are encompassed herein as well as methods for producing these.

In one aspect, such progeny have all the physiological and morphological characteristics of pepper variety NUN 70030 PPH when grown under the same environmental conditions. In another aspect such progeny have all the physiological and morphological characteristics as listed in Table 1 as pepper variety NUN 70030 PPH when measured under the same environmental conditions (i.e., evaluated at significance levels of 1%, 5% or 10% significance, which can also be expressed as a p value).

In another aspect a plant of the invention or said progeny plants has/have 1, 2, 3, 4 or more or all of the distinguishing characteristics: 1) an average of 67.7 to 74.76 fruits per plant; 2) heat of (dried) fruit expressed as Scoville units of 22610 to 24990; 3) maturity (at region of adaptability), expressed as days from transplanting until mature red stage of 85.5 to 94.5; 4) an average fruit diameter at mid-point of 36.3 to 40.1 mm; 5) an average fruit length of 95.0 to 105.0 mm; 6) an average stem length from cotyledons to first flower of 23.7 to 26.1 cm; 7) a concentrated fruit set; 8) an average of 128.5 to 142.0 seeds per fruit; 9) an average seed cavity length of 84.0 to 92.8 mm and 10) an average leaf width of 44.9 to 49.7 mm, in addition to 3, 4, 5, 6, 7, 8, or more, or all of the other (average) characteristics as listed in Table 1.

Further, a pepper fruit produced on a plant grown from these seeds is provided.

In yet another embodiment of the invention, a plant having one, two or three physiological and/or morphological characteristics which are different from those of NUN 70030 PPH and which otherwise has all the physiological and morphological characteristics of NUN 70030 PPH as listed in Table 1, wherein a representative sample of seed of variety NUN 70030 PPH has been deposited under Accession Number NCIMB ______, is provided.

Further, a vegetatively propagated plant of variety NUN 70030 PPH, or a part thereof, is provided having all the morphological and physiological characteristics of NUN 70030 PPH when grown under the same environmental conditions.

Also a plant part derived from variety NUN 70030 is provided, wherein said plant part is selected from the group consisting of: fruit, harvested fruit, parts of fruits, leaf, pollen, ovule, cell, part of a leaf, petioles, shoots or parts thereof, stems or parts thereof, roots or parts thereof, cuttings, seeds, hypocotyl, cotyledon, flowers or parts thereof, scion, cion, stock, rootstock and flower. Fruits are particularly important plant parts.

### DEFINITIONS

“Pepper” refers herein to plants of the species Capsicum annuum or frutescens, and fruits thereof.

“Cultivated pepper” refers to plants of Capsicum annuum or frutescens i.e., varieties, breeding lines or cultivars of the species C. annuum or frutescens as well as crossbreds thereof, or crossbreds with other Capsicum species, cultivated by humans and having good agronomic characteristics; preferably such plants are not “wild plants”, i.e., plants which generally have much poorer yields and poorer agronomic characteristics than cultivated plants and e.g., grow naturally in wild populations. “Wild plants” include for example ecotypes, PI (Plant Introduction) lines, landraces or wild accessions or wild relatives of Capsicum and related species.

The terms “pepper plant designated NUN 70030”, “Emperador”, “NUN 70030” “70030 PPH” or “variety designated NUN 70030” are used interchangeably herein and refer to a pepper plant of variety NUN 70030 PPH, representative seed of which having been deposited under Accession Number NCIMB ______.

“Tissue culture” refers to a composition comprising isolated cells of the same or a different type or a collection of such cells organized into parts of a plant. Tissue culture of various tissues of pepper and regeneration of plants therefrom is well known and widely published (see, e.g., Sang-Gu et al. (1988), Plant Cell, Tissue and Organ Culture 12: 67-74; Kothari et al., (2010) Biotechnology Advances 28: 35-48. Similarly, the skilled person is well-aware how to prepare a “cell culture”.

“UPOV descriptors” are the plant variety descriptors described for pepper in the “Guidelines for the Conduct of Tests for Distinctness, Uniformity and Stability, TG/76/8 (Geneva, 2006), as published by UPOV (International Union for the Protection of New Varieties and Plants, available on the world wide web at upov.int) and which can be downloaded from the world wide web at upov.int/ under edocs/tgdocs/en/tg076.pdf and is herein incorporated by reference in its entirety.

“USDA descriptors” are the plant variety descriptors for pepper (Capsicum spp.) as published by the US Department of Agriculture, Agricultural Marketing Service, Plant Variety Protection Office, Beltsville, Md. 20705 (available on the world wide web at ams.usda.gov) and which can be downloaded from the world wide web at ams.usda.gov/AMSv1.0/getfile?dDocName=STELDEV3003768.

“RHS” refers to the Royal Horticultural Society of England which publishes an official botanical color chart quantitatively identifying colors according to a defined numbering system. The chart may be purchased from Royal Horticulture Society Enterprise Ltd RHS Garden; Wisley, Woking; Surrey GU236QB, UK, e.g., the RHS colour chart: 2007 (The Royal Horticultural Society, charity No: 222879, PO Box 313 London SW1P2PE; sold by, e.g., TORSO-VERLAG, Obere Grüben 8•D-97877 Wertheim, Article-No.: Art62-00008 EAN-Nr.: 4250193402112).

As used herein, the term “plant” includes the whole plant or any parts or derivatives thereof, preferably having the same genetic makeup as the plant from which it is obtained, such as plant organs (e.g., harvested or non-harvested fruits), plant cells, plant protoplasts, plant cell tissue cultures or tissue cultures from which whole plants can be regenerated, plant calli, plant cell clumps, plant transplants, seedlings, hypocotyl, cotyledon, plant cells that are intact in plants, plant clones or micropropagations, or parts of plants (e.g., harvested tissues or organs), such as plant cuttings, vegetative propagations, embryos, pollen, ovules, fruits, flowers, leaves, seeds, clonally propagated plants, roots, stems, root tips, grafts, scions, rootstocks, parts of any of these and the like. Also any developmental stage is included, such as seedlings, cuttings prior or after rooting, mature plants or leaves.

“Harvested plant material” refers herein to plant parts (e.g., fruits detached from the whole plant) which have been collected for further storage and/or further use.

“Harvested seeds” refers to seeds harvested from a line or variety, e.g., produced after self-fertilization or cross-fertilization and collected.

“Internode” refers to a portion of a plant stem between nodes.

“Node” refers to the place on a plant stem where a leaf is attached.

A plant having “all the physiological and morphological characteristics” of a referred-to-plant means a plant having the physiological and morphological characteristics of the referred-to-plant when grown under the same environmental conditions; the referred-to-plant can be a plant from which it was derived, e.g., the progenitor plant, the parent, the recurrent parent, the plant used for tissue- or cell culture, etc.

A plant having “essentially all the physiological and morphological characteristics” of a referred-to-plant means a plant having all the (e.g., 1-10) distinguishing physiological and morphological characteristics (distinguishing characteristics as herein defined) when grown under the same environmental conditions of the referred-to-plant (e.g., a plant from which it was derived such as the progenitor plant, the parent, the recurrent parent, the plant used for tissue- or cell culture, etc.) Alternatively, a plant having “essentially all the physiological and morphological characteristics” of a referred-to-plant means a plant having all the characteristics as listed in Table 1 when grown under the same environmental conditions as a referred-to-plant (e.g., a plant from which it was derived such as the progenitor plant, the parent, the recurrent parent, the plant used for tissue- or cell culture, etc.). In another embodiment, a plant having “essentially all the physiological and morphological characteristics” of a referred-to-plant means a plant having all but 1, 2, 3, 4 or 5 of the characteristics as listed in Table 1 when grown under the same environmental conditions as a referred-to-plant (e.g., a plant from which it was derived such as the progenitor plant, the parent, the recurrent parent, the plant used for tissue- or cell culture, etc.).

For NUN 70030 PPH the distinguishing characteristics are 1) average number of fruits per plant; 2) heat of (dried) fruit expressed as Scoville units; 3) maturity (at region of adaptability), expressed as days from transplanting until mature red stage; 4) average fruit diameter at mid-point; 5) average fruit length; 6) average stem length from cotyledons to first flower; 7) a concentrated fruit set; 8) average number of seeds per fruit; 9) average seed cavity length and 10) average leaf width.

In certain embodiments the plant of the invention has all the physiological and morphological characteristics, except for certain characteristics mentioned, e.g., the characteristic(s) derived from a converted or introduced gene or trait and/or except for the characteristics which differ.

Similarity between different plants is defined as the number of distinguishing characteristics (or the characteristics as listed in Table 1) that are the same between the two plants that are compared when grown under the same environmental conditions. Characteristics are considered “the same” when the value for a numeric characteristic is evaluated at significance levels of 1%, 5% or 10% significance level, or when a non-numeric characteristic is identical, if the plants are grown under the same conditions.

A plant having one or more “essential physiological and/or morphological characteristics” or one or more “distinguishing characteristics” refers to a plant having (or retaining) one or more of the characteristics mentioned in Table 1 when grown under the same environmental conditions that distinguish NUN 7030 PPH from the most similar varieties (such as variety Piton), such as but not limited to average number of fruits per plant, heat of (dried) fruit, maturity, average fruit diameter at mid-point or average fruit length.

“Distinguishing characteristics” or “distinguishing morphological and/or physiological characteristics” refers herein the characteristics which are distinguishing between NUN 70030 PPH and other pepper varieties, such as Piton, when grown under the same environmental conditions, especially the following characteristics: 1) an average of 71.2 fruits per plant; 2) heat of (dried) fruit expressed as Scoville units of 23800; 3) maturity (at region of adaptability), expressed as days from transplanting until mature red stage of 90; 4) an average fruit diameter at mid-point of 38.2 mm; 5) an average fruit length of 100.0 mm; 6) an average stem length from cotyledons to first flower of 24.9 cm; 7) a concentrated fruit set; 8) an average of 135.2 seeds per fruit; 9) an average seed cavity length of 88.4 mm and 10) an average leaf width of 47.3 mm. In one aspect, the distinguishing characteristics further include at least one, two, three or more (or all) of the characteristics listed in Table 1. All numerical distinguishing characteristics are statistically significantly different at p≦0.05.

Thus, a pepper plant “comprising the distinguishing characteristics of NUN 70030 PPH” refers herein to a pepper plant which does not differ significantly from NUN 70030 PPH in characteristics 1) to 5) above. In a further aspect the pepper plant further does not differ significantly from NUN 70030 PPH in one or more, or all characteristics 6) to 10) as mentioned above. In yet a further aspect the pepper plant further does not differ in at least one, two, three, four, five or six (or all) characteristics selected from the characteristics listed in Table 1. In still another aspect the pepper plant does not differ in any of the distinguishing characteristics 1) to 10) listed above.

The physiological and/or morphological characteristics mentioned above are commonly evaluated at significance levels of 1%, 5% or 10%, when measured under the same environmental conditions. For example, a progeny plant of NUN 70030 PPH may have one or more (or all) of the essential physiological and/or morphological characteristics of NUN 70030 PPH listed in Table 1, as determined at the 5% significance level when grown under the same environmental conditions.

As used herein, the term “variety” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

A variety is referred to as an “Essentially Derived Variety” (EDV) i.e., shall be deemed to be essentially derived from another variety, “the initial variety” when (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and (iii) except for the differences which result from the act of derivation, it conforms to the initial variety in the expression of the essential characteristics that result from the genotype or combination of genotypes of the initial variety. Thus, an EDV may be obtained for example by the selection of a natural or induced mutant, or of a somaclonal variant, the selection of a variant individual from plants of the initial variety, backcrossing, or transformation by genetic engineering.

“Plant line” is for example a breeding line which can be used to develop one or more varieties. Progeny obtained by selfing a plant line has the same phenotype as its parents.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent.

“Regeneration” refers to the development of a plant from cell culture or tissue culture or vegetative propagation.

“Vegetative propagation”, “vegetative reproduction” or “clonal propagation” are used interchangeably herein and mean the method of taking part of a plant and allowing that plant part to form at least roots where plant part is, e.g., defined as or derived from (e.g., by cutting of) leaf, pollen, embryo, cotyledon, hypocotyl, cells, protoplasts, meristematic cell, root, root tip, pistil, anther, flower, shoot tip, shoot, stem, fruit, petiole, etc. When a whole plant is regenerated by vegetative propagation, it is also referred to as a vegetative propagation.

“Selfing” refers to self-pollination of a plant, i.e., the transfer of pollen from the anther to the stigma of the same plant. “Crossing” refers to the mating of two parent plants.

“Average” refers herein to the arithmetic mean.

“Substantially equivalent” refers to a characteristic that, when compared, does not show a statistically significant difference (e.g., p=0.05) from the mean. ANOVA is a suitable method for determining the value of p (Clewer, A. G., and D. H. Scarisbrick. 2001).

“Locus” (plural loci) refers to the specific location of a gene or DNA sequence on a chromosome. A locus may confer a specific trait.

“Allele” refers to one or more alternative forms of a gene locus. All of these loci relate to one trait. Sometimes, different alleles can result in different observable phenotypic traits, such as different pigmentation. However, many variations at the genetic level result in little or no observable variation. If a multicellular organism has two sets of chromosomes, i.e., diploid, these chromosomes are referred to as homologous chromosomes. Diploid organisms have one copy of each gene (and therefore one allele) on each chromosome. If both alleles are the same, they are homozygotes. If the alleles are different, they are heterozygotes.

“Genotype” refers to the genetic composition of a cell or organism.

“Maturity” refers to the fruit developmental stage when the fruit has fully developed (reached its final size), begins to ripen and undergoes ripening, during which fruits can be divided into 1, 2, 3 or more maturity stages. Thereafter, fruits become overripe. In particular embodiments “maturity” is defined as the mature stage of fruit development and optimal time for harvest. In one embodiment a “mature” pepper is defined as having reached the stage of maturity which will insure the proper completion of the normal ripening process. In particular embodiments, fruit should be harvested at a maturity stage i.e., substantially near maximum sweetness and flavor intensity.

“Harvest maturity” is referred to as the stage at which a pepper fruit is ripe or ready for harvest or the optimal time to harvest the fruit. In one embodiment, harvest maturity is the stage which allows proper completion of the normal ripening.

“Flavor” refers to the sensory impression of a food or other substance, especially a pepper fruit or fruit part (fruit flesh) and is determined mainly by the chemical senses of taste and smell. Flavor is influenced by texture properties and by volatile and/or non-volatile chemical components (organic acids, lipids, carbohydrates, salts etc.)

The term “traditional breeding techniques” encompasses herein crossing, selfing, selection, double haploid production, embryo rescue, protoplast fusion, marker assisted selection, mutation breeding etc. as known to the breeder (i.e., methods other than genetic modification/transformation/transgenic methods), by which, for example, a genetically heritable trait can be transferred from one pepper line or variety to another.

“Backcrossing” is a traditional breeding technique used to introduce a trait into a plant line or variety. The plant containing the trait is called the donor plant and the plant into which the trait is transferred is called the recurrent parent. An initial cross is made between the donor parent and the recurrent parent to produce progeny plants. Progeny plants which have the trait are then crossed to the recurrent parent. After several generations of backcrossing and/or selfing the recurrent parent comprises the trait of the donor. The plant generated in this way may be referred to as a “single trait converted plant”. “Progeny” as used herein refers to plants derived from a plant designated NUN 70030 PPH. Progeny may be derived by regeneration of cell culture or tissue culture or parts of a plant designated NUN 70030 PPH or selfing of a plant designated NUN 70030 PPH or by producing seeds of a plant designated NUN 70030 PPH. In further embodiments, progeny may also encompass plants derived from crossing of at least one plant designated NUN 70030 PPH with another pepper plant of the same or another variety or (breeding) line, or wild pepper plants, backcrossing, inserting of a locus into a plant or mutation. A progeny is, e.g., a first generation progeny, i.e., the progeny is directly derived from, obtained from, obtainable from or derivable from the parent plant by, e.g., traditional breeding methods (selfing and/or crossing) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above.

The terms “gene converted” or “conversion plant” in this context refer to pepper plants which are developed by backcrossing wherein essentially all of the desired morphological and physiological characteristics of parent are recovered in addition to the one or more genes transferred into the parent via the backcrossing technique or via genetic engineering. Likewise a “Single Locus Converted (Conversion) Plant” refers to plants which are developed by plant breeding techniques comprising or consisting of backcrossing, wherein essentially all of the desired morphological and physiological characteristics of a pepper variety are recovered in addition to the characteristics of the single locus having been transferred into the variety via the backcrossing technique and/or by genetic transformation.

“Transgene” or “chimeric gene” refers to a genetic locus comprising a DNA sequence which has been introduced into the genome of a pepper plant by transformation. A plant comprising a transgene stably integrated into its genome is referred to as “transgenic plant”.

The term “mean” refers to the arithmetic mean of several measurements. The skilled person understands that the appearance of a plant depends to some extent on the growing conditions of said plant. Thus, the skilled person will know typical growing conditions for peppers described herein. The mean, if not indicated otherwise within this application, refers to the arithmetic mean of measurements on at least 10 different, randomly selected plants of a variety or line.

“Substantially equivalent” refers to a characteristic that, when compared, does not show a statistically significant difference (e.g., p>0.05) from the mean.

Scoville Heat Unit (SHU) or Scoville Scale or Scoville Units is measurement of the pungency (spicy heat) of peppers as reported in Scoville heat units (SHU), a function of capsaicin concentration. The scale is named after its creator, American pharmacist Wilbur Scoville. The Scoville scale can be measured empirically, dependent on the capsaicin sensitivity of testers (i.e., by tasting the pepper or tasting diluted pepper samples until heat no longer can be detected by the tasters. Alternatively, high-performance liquid chromatography (HPLC) can used to determine SHU. In this procedure, fruits are dried and then ground, next, the chemicals responsible for heat are extracted, and the extract is injected into the HPLC for analysis. SHU of NUN 70030 PPH has been determined using HPLC.

### DETAILED DESCRIPTION

The present invention relates to a Capsicum spp. variety, referred to as NUN 70030 PPH, which has a higher average number of fruits per plant, greater heat of (dried) fruit, earlier maturity, larger average fruit diameter at mid-point, shorter average fruit length, longer average stem length from cotyledons to first flower, concentrated fruit set instead of scattered fruit set, lower average number of seeds per fruit, shorter average seed cavity length and narrower average leaf width than check variety Piton. Also encompassed by the present invention are progeny plants having all but 1, 2, or 3 of the morphological and/physiological characteristics of NUN 70030 PPH and methods of producing plants in accordance with the present invention.

A pepper plant of NUN 70030 PPH differs from the most similar comparison variety Piton in one or more characteristics (referred herein to as “distinguishing characteristics” or “distinguishing morphological and/or physiological characteristics” (or essential physiological and/or morphological characteristics) selected from

1) NUN 70030 PPH has an average of number of fruits per plant that is at least 5%, or preferably 7, 9, 10, 11, 12% or even more preferably about 12.7% higher than Piton;
2) NUN 70030 PPH has heat of (dried) fruit expressed as Scoville units that is at least 100%, or preferably 150, 200, 250, 275, 290% or even more preferably about 298% higher than Piton;
3) NUN 70030 PPH has maturity (at region of adaptability), expressed as days from transplanting until mature red stage that is at least 5%, or preferably 6, 7, 8, 9% or even more preferably about 10% earlier than Piton;
4) NUN 70030 PPH has an average fruit diameter at mid-point that is at least 4%, or preferably 5, 5.5, 6, 6.5% or even more preferably about 7% higher than Piton;
5) NUN 70030 PPH has an average fruit length that is at least 5%, or preferably 6, 7, 8, 9% or even more preferably about 10.1% shorter than Piton;
6) NUN 70030 PPH has an average stem length from cotyledons to first flower that is at least 5%, or preferably 7, 9, 11, 12% or even more preferably about 13.7% longer than Piton;
7) NUN 70030 PPH has a concentrated fruit set instead of a scattered fruit set which Piton has;
8) NUN 70030 PPH has an average number of seeds per fruit that is at least 10%, or preferably 15, 20, 25, 27% or even more preferably about 29% lower than Piton;
9) NUN 70030 PPH has an average seed cavity length that is at least 2%, or preferably 2, 3, 4% or even more preferably about 4.9% shorter than Piton; and
10) NUN 70030 PPH has an average leaf width that is at least 10%, or preferably 15, 20, 22, 24% or even more preferably about 24.3% longer than Piton.

It is understood that “significant” differences refer to statistically significant differences, when comparing the characteristic between two plant lines or varieties when grown under the same conditions. Preferably at least about 10, 15, 20 or more plants per line or variety are grown under the same conditions (i.e., side by side) and characteristics are measured on at least about 10, 15, 20 or more randomly selected plant or plant parts to obtain averages. Thus, physiological and morphological characteristics or traits are commonly evaluated at a significance level of 1%, 5% or 10%, when measured in plants grown under the same environmental conditions.

Thus, in one aspect, the invention provides seeds of the pepper variety designated NUN 70030 PPH wherein a representative sample of seeds of said variety was deposited under the Budapest Treaty, with Accession number NCIMB ______.

Seeds of NUN 70030 PPH are obtainable by crossing the male parent with the female parent and harvesting the seeds produced on the female parent. The resultant NUN 70030 PPH seeds can be grown to produce NUN 70030 PPH plants. In one embodiment a plurality of NUN 70030 PPH seeds are packaged into small and/or large containers (e.g., bags, cartons, cans, etc.). The seeds may be disinfected, primed and/or treated with various compounds, such as seed coatings or crop protection compounds.

Also provided are plants of pepper variety NUN 70030 PPH, or a fruit or other plant part thereof, produced from seeds, wherein a representative sample of said seeds has been deposited under the Budapest Treaty, with Accession Number NCIMB ______. Also included is a cell culture or tissue culture produced from such a plant or a plant regenerated from such a cell or tissue culture said plant expressing all the morphological and physiological characteristics of NUN 70030 PPH.

Plants of NUN 70030 PPH can be produced by seeding directly in the ground (e.g., field) or by germinating the seeds in controlled environment conditions (e.g., greenhouses) and then transplanting the seedlings into the field. For example, the seed can be sown into prepared seed beds where they will remain for the entire production of the crop. Alternatively, the pepper seed may be planted through a black plastic mulch. The dark plastic will absorb heat from the sun, warming the soil early. It will also help to conserve moisture during the growing season, controls weeds and makes harvesting easier and cleaner. Pepper can also be grown entirely in greenhouses. See for example, J. Burt, Farmnote 64/99 for cultivation, harvesting, handling and postharvest methods commonly used.

In another aspect, the invention provides for a pepper plant of variety NUN 70030 PPH, a representative sample of seed from said variety has been deposited under the Budapest Treaty, with Accession number NCIMB ______.

In other aspects, the invention provides for a fruit of pepper variety NUN 70030 PPH, or a plant part, such as pollen, flowers, shoots or cuttings of variety NUN 70030 PPH or parts thereof.

In one embodiment any plant of the invention comprises at least 3, 4, 5 or more, e.g., 6, 7, 8, 9 or all of the following morphological and/or physiological characteristics (i.e., distinguishing characteristics (average values; measured at harvest or market maturity, as indicated on the USDA Objective description of variety—Pepper (unless indicated otherwise), when grown under the same environmental conditions):

1) NUN 70030 PPH has an average of number of fruits per plant of about 71.2 e.g., between about 50 and about 90 or preferably between about 60 and about 80 or between about 65 and 75 or even between about 71 and 72 fruits;
2) NUN 70030 PPH has heat of (dried) fruit expressed as Scoville units that is about 23800 e.g., between about 15000 and about 30000 or preferably between about 20000 and about 25000 or between about 22000 and 24000 or even between about 23600 and 23900;
3) NUN 70030 PPH has maturity (at region of adaptability), expressed as days from transplanting until mature red stage of about 90 days e.g., between about 75 and about 105 days or preferably between about 80 and about 100 days or between about 85 and 95 days or even between about 88 and 92 days;
4) NUN 70030 PPH has an average fruit diameter at mid-point of about 38.2 mm e.g., between about 30 mm and about 50 mm or preferably between about 35 mm and about 45 mm or between about 37 mm and 40 mm or even between about 38 mm and 39 mm;
5) NUN 70030 PPH has an average fruit length of about 100.0 mm e.g., between about 80 mm and about 120 mm or preferably between about 90 mm and about 110 mm or between about 95 mm and 105 mm or even between about 98 mm and 102 mm;
6) NUN 70030 PPH has an average stem length from cotyledons to first flower of about 249 mm e.g., between about 200 mm and about 300 mm or preferably between about 225 mm and about 275 mm or between about 240 mm and 260 mm or even between about 245 mm and 255 mm;
7) NUN 70030 PPH has a concentrated fruit set;
8) NUN 70030 PPH has an average number of seeds per fruit of about 135.2 seeds e.g., between about 110 and about 160 or preferably between about 120 and about 150 or between about 130 and 140 or even between about 133 and 137 seeds;
9) NUN 70030 PPH has an average seed cavity length of about 88.4 mm e.g., between about 200 mm and about 300 mm or preferably between about 225 mm and about 275 mm or between about 95 mm and 105 mm or even between about 87 mm and 90 mm; and
10) NUN 70030 PPH has an average leaf width of about 47.3 mm e.g., between about 35 mm and about 60 mm or preferably between about 40 mm and about 55 mm or between about 45 mm and 50 mm or even between about 47 mm and 48 mm.

In another embodiment, NUN 70030 PPH has intermediate to high resistance against the viral disease Potato Y virus and the bacterial diseases Bacterial Spot (Xanthomonas vesicatoria) and Cercospora Leaf Spot (Cercospora capsici). Resistance against Potato Y virus is classified as intermediate to high (7 out of 9, with 9 highest), resistance against Bacterial Spot is classified as high (8 out of 9) and resistance against Cercospora Leaf Spot is classified as intermediate (5 out of 9). All Rates are from 1 (most susceptible) to 9 (most resistant).

In still another aspect the invention provides a method of producing a pepper plant, comprising crossing a plant of pepper variety NUN 70030 PPH with a second pepper plant one or more times, and selecting progeny from said crossing.

In yet another aspect the invention provides a method of producing a pepper plant, comprising selfing a plant of pepper variety NUN 70030 PPH one or more times, and selecting progeny from said selfing.

In other aspects, the invention provides for progeny of variety NUN 70030 PPH such as progeny obtained by further breeding NUN 70030 PPH. Further breeding NUN 70030 PPH includes selfing NUN 70030 PPH one or more times and/or cross-pollinating NUN 70030 PPH with another pepper plant or variety one or more times. In particular, the invention provides for progeny that retain all the essential morphological and physiological characteristics of NUN 70030 PPH or that retain one or more of the distinguishing characteristics of the pepper type described further above and when grown under the same environmental conditions. In another aspect, the invention provides for vegetative reproductions of the variety and plants having all but 1, 2, or 3 of the physiological and morphological characteristics of NUN 70030 PPH (e.g., as listed in Table 1).

The morphological and/or physiological differences between plants according to the invention, i.e., NUN 70030 PPH or progeny thereof, or plants having all but 1, 2, or 3 of the physiological and morphological characteristics of NUN 70030 PPH (as listed in Table 1); and other known varieties can easily be established by growing NUN 70030 PPH next to the other varieties (in the same field, under the same environmental conditions), preferably in several locations which are suitable for said pepper cultivation, and measuring morphological and/or physiological characteristics of a number of plants (e.g., to calculate an average value and to determine the variation range/uniformity within the variety). For example, trials can be carried out in Acampo Calif., USA (N 38 degrees 07′261″/W 121 degrees 18′ 807″, USA, whereby, maturity, days from seeding to harvest, plant habit, plant attitude, leaf shape, leaf color, blistering, numbers of flowers per leaf axil, number of calyx lobes, number of petals, fruit group, immature fruit color, mature fruit color, pungency, flavor, fruit glossiness, fruit size, fruit shape, average number of fruits per plant, seed size, seed weight, anthocyanin level, disease resistance, insect resistance, can be measured and directly compared for species of Capsicum.

The morphological and physiological characteristics (and distinguishing characteristics) of NUN 70030 PPH, are provided in the Examples, in Table 1. Encompassed herein are also plants derivable from NUN 70030 PPH (e.g., by selfings and/or crossing and/or backcrossing with NUN 70030 PPH and/or progeny thereof) comprising all the physiological and morphological characteristics of NUN 70030 PPH listed in Table 1 as determined at the 5% significance level when grown under the same environmental conditions and/or comprising one or more (or all; or all except one, two or three) of the distinguishing characteristics as determined at the 5% significance level when grown under the same environmental conditions.

Also at-harvest and/or post-harvest characteristics of fruits can be compared, such as cold storage holding quality (browning), post-harvest rind firmness and/or flesh firmness, can be measured using known methods.

Flesh firmness can for example be measured using a penetrometer, e.g., by inserting a probe into the fruit flesh and determining the insertion force, or by other methods.

The morphological and/or physiological characteristics may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (World wide web at rhs.org.uk/Plants/RHS-Publications/RHS-colour-charts).

In a preferred embodiment, the invention provides for pepper fruits of variety NUN 70030 PPH, or a part of the fruit. In another embodiment, the invention provides for a container comprising or consisting of a plurality of harvested pepper fruits of NUN 70030 PPH, or progeny thereof, or a derived variety.

In yet a further embodiment, the invention provides for a method of producing a new pepper plant. The method comprises crossing a plant of the invention NUN 70030 PPH, or a plant comprising all but 1, 2, or 3 of the morphological and physiological characteristics of NUN 70030 PPH (as listed in Table 1), or a progeny plant thereof, either as male or as female parent, with a second pepper plant (or a wild relative of pepper) one or more times, and/or selfing a pepper plant according to the invention i.e., NUN 70030 PPH, or a progeny plant thereof, one or more times, and selecting progeny from said crossing and/or selfing. The second pepper plant may for example be a line or variety of the species Capsicum annuum, C. frutecens, C. baccatum, C. chinense, or other Capsicum species.

Progeny are either the generation (seeds) produced from the first cross (F1) or selfing (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g., the F2) with another pepper plant (and/or with a wild relative of pepper). Progeny may have all the physiological and morphological characteristics of pepper variety NUN 70030 PPH when grown under the same environmental conditions and/or progeny may have (be selected for having) one or more of the distinguishing characteristics of pepper of the invention. Using common breeding methods such as backcrossing or recurrent selection, one or more specific characteristics may be introduced into NUN 70030 PPH, to provide or a plant comprising all but 1, 2, or 3 or more of the morphological and physiological characteristics of NUN 70030 PPH (as listed in Table 1).

The invention provides for methods of producing plants which retain all the morphological and physiological characteristics of NUN 70030 PPH. The invention provides also for methods of producing a plant comprising all but 1, 2, or 3 or more of the morphological and physiological characteristics of NUN 70030 PPH (e.g., as listed in Table 1), but which are still genetically closely related to NUN 70030 PPH. The relatedness can, for example be determined by fingerprinting techniques (e.g., making use of isozyme markers and/or molecular markers such as SNP markers, AFLP markers, microsatellites, minisatellites, RAPD markers, RFLP markers and others). A plant is “closely related” to NUN 70030 PPH if its DNA fingerprint is at least 80%, 90%, 95% or 98% identical to the fingerprint of NUN 70030 PPH. In a preferred embodiment AFLP markers are used for DNA fingerprinting (Vos et al. 1995, Nucleic Acid Research 23: 4407-4414). A closely related plant may have a Jaccard's Similarity index of at least about 0.8, preferably at least about 0.9, 0.95, 0.98 or more (Ince et al., (2010) Biochem. Genet. 48:83-95). The invention also provides plants and varieties obtained by these methods. Plants may be produced by crossing and/or selfing, or alternatively, a plant may simply be identified and selected amongst NUN 70030 PPH plants, or progeny thereof, e.g., by identifying a variant within NUN 70030 PPH or progeny thereof (e.g., produced by selfing) which variant differs from NUN 70030 PPH in one, two or three of the morphological and/or physiological characteristics (e.g., in one, two or three distinguishing characteristics), e.g., those listed in Table 1 or others.

By crossing and/or selfing also (one or more) single traits may be introduced into the variety of the invention i.e., NUN 70030 PPH (e.g., using backcrossing breeding schemes), while retaining the remaining morphological and physiological characteristics of NUN 70030 PPH and/or while retaining one or more distinguishing characteristics. A single trait converted plant may thereby be produced. For example, disease resistance genes may be introduced, genes responsible for one or more quality traits, yield, etc. Both single genes (dominant or recessive) and one or more QTLs (quantitative trait loci) may be transferred into NUN 70030 PPH by breeding with NUN 70030 PPH.

Any pest or disease resistance genes may be introduced into a plant according to the invention, i.e., NUN 70030 PPH, progeny thereof or into a plant comprising all but 1, 2, or 3 or more of the morphological and physiological characteristics of NUN 70030 PPH (e.g., as listed in Table 1). Resistance to one or more of the following diseases is preferably introduced into plants of the invention: Cucumber Mosaic Virus, Curly Top Virus, Pepper Mottle Virus, Potato Y Virus, Tobacco Etch Virus, Tobacco Mosaic Virus, Anthracnose (Gloeosporium piperatum), Bacterial Spot (Xanthomonas vesicatoria), Cercospora Leaf Spot (Cercospora capsici), Nematode (Meloidogyne incognita acrita), Phytophthora Root Rot (Phytophthora capsici), Ripe Rot (Vermicularia capsici), Southern Blight (Sclerotium rolfsii) and/or Verticillium Wilt (Verticillium dahliae).

Other resistance genes, against pathogenic viruses, fungi, bacteria, nematodes, insects or other pests may also be introduced.

Thus, invention also provides a method for developing a pepper plant in a pepper breeding program, using a pepper plant of the invention, or its parts as a source of plant breeding material. Suitable plant breeding techniques are recurrent selection, backcrossing, pedigree breeding, mass selection, mutation breeding and/or genetic marker enhanced selection. For example, in one aspect, the method comprises crossing NUN 70030 PPH or progeny thereof, or a plant comprising all but 1, 2, or 3 or more of the morphological and physiological characteristics of NUN 70030 PPH (e.g., as listed in Table 1), with a different pepper plant, and wherein one or more offspring of the crossing are subject to one or more plant breeding techniques selected from the group consisting of recurrent selection, backcrossing, pedigree breeding, mass selection, mutation breeding and genetic marker enhanced selection (see e.g., Thabuis et al., (2004) Theor Appl Genet 109:342-351). For breeding methods in general see Principles of Plant Genetics and Breeding, 2007, George Acquaah, Blackwell Publishing, ISBN-13: 978-1-4051-3646-4.

The invention thus also provides a method of introducing a single locus conversion, or single trait conversion or introducing a desired trait, into a pepper plant according to the invention and/or into NUN 70030 PPH comprising:
(a) crossing a pepper plant of variety NUN 70030 PPH, a representative sample of seed of said variety having been deposited under Accession Number NCIMB ______, with a second pepper plant comprising a desired single locus to produce F1 progeny plants;
(b) selecting F1 progeny plants that have the single locus to produce selected F1 progeny plants;
(c) crossing the selected progeny plants with a plant of NUN 70030 PPH, to produce backcross progeny plants;
(d) selecting backcross progeny plants that have the single locus and one or more (or all) distinguishing characteristics of pepper according to the invention and/or all the physiological and morphological characteristics of NUN 70030 PPH to produce selected backcross progeny plants; and
(e) optionally repeating steps (c) and (d) one or more times in succession to produce selected second, third or fourth or higher backcross progeny plants that comprise the single locus and otherwise one or more (or all) the distinguishing characteristics of the peppers according to the invention and/or comprise all of the physiological and morphological characteristics of NUN 70030 PPH, when grown in the same environmental conditions. The invention further relates to plants obtained by this method.

The above method is provided, wherein the single locus confers a trait, wherein the trait is pest resistance or disease resistance.

In one embodiment the trait is disease resistance and the resistance is conferred to Cucumber Mosaic Virus, Curly Top Virus, Pepper Mottle Virus, Potato Y Virus, Tobacco Etch Virus, Tobacco Mosaic Virus, Anthracnose (Gloeosporium piperatum), Bacterial Spot (Xanthomonas vesicatoria), Cercospora Leaf Spot (Cercospora capsici), Nematode (Meloidogyne incognita acrita), Phytophthora Root Rot (Phytophthora capsici), Ripe Rot (Vermicularia capsici), Southern Blight (Sclerotium rolfsii), and Verticillium Wilt (Verticillium dahliae).

The invention also provides a pepper plant comprising at least a first set of the chromosomes of pepper variety NUN 70030 PPH, a sample of seed of said variety having been deposited under Accession Number NCIMB ______; optionally further comprising a single locus conversion, wherein said plant has essentially all of the morphological and physiological characteristics of the plant comprising at least a first set of the chromosomes of pepper NUN 70030 PPH. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

In one embodiment, NUN 70030 PPH may also be mutated (by e.g., irradiation, chemical mutagenesis, heat treatment, etc.) and mutated seeds or plants may be selected in order to change one or more characteristics of NUN 70030 PPH. Methods such as TILLING may be applied to pepper populations in order to identify mutants. Similarly, NUN 70030 PPH may be transformed and regenerated, whereby one or more chimeric genes are introduced into the variety or into a plant comprising all but 1, 2, 3, or more of the morphological and physiological characteristics (e.g., as listed in Table 1). Transformation can be carried out using standard methods, such as Agrobacterium tumefaciens mediated transformation or biolistics, followed by selection of the transformed cells and regeneration into plants. A desired trait (e.g., genes conferring pest or disease resistance, herbicide, fungicide or insecticide tolerance, etc.) can be introduced into NUN 70030 PPH, or progeny thereof, by transforming NUN 70030 PPH or progeny thereof with a transgene that confers the desired trait, wherein the transformed plant retains all the phenotypic and/or morphological and/or physiological characteristics of NUN 70030 PPH or the progeny thereof and contains the desired trait.

The invention also provides for progeny of pepper variety NUN 70030 PPH obtained by further breeding with NUN 70030 PPH. In one aspect progeny are F1 progeny obtained by crossing NUN 70030 PPH with another plant or 51 progeny obtained by selfing NUN 70030 PPH. Also encompassed are F2 progeny obtained by selfing the F1 plants. “Further breeding” encompasses traditional breeding (e.g., selfing, crossing, backcrossing), marker assisted breeding, and/or mutation breeding. In one embodiment, the progeny have one or more (or all) of the distinguishing characteristics mentioned further above when grown under the same environmental conditions. In a further embodiment the progeny have all the physiological and morphological characteristics of variety NUN 70030 PPH when grown under the same environmental conditions. In another embodiment the progeny have one, two, or three distinct traits (qualitative or quantitative) introduced into NUN 70030 PPH, while retaining all the other physiological and morphological characteristics of variety NUN 70030 PPH when grown under the same environmental conditions.

An EDV is a plant having one, two or three physiological and/or morphological characteristics which are different from those of NUN 70030 PPH and which otherwise has all the physiological and morphological characteristics of NUN 70030 PPH, wherein a representative sample of seed of variety NUN 70030 PPH has been deposited under Accession Number NCIMB ______. In particular variants which differ from NUN 70030 PPH in none, one, two or three of the characteristics mentioned in Table 1 are encompassed.

In one aspect, the EDV differs from NUN 70030 PPH in one, two or three of the distinguishing morphological and/or physiological characteristics selected from 1) average number of fruits per plant; 2) heat of (dried) fruit expressed as Scoville units; 3) maturity (at region of adaptability), expressed as days from transplanting until mature red stage; 4) average fruit diameter at mid-point; 5) average fruit length.

In another embodiment the EDV may differ from NUN 70030 PPH in one, two or three morphological or physiological characteristic other than the “distinguishing morphological and/or physiological characteristics” (or essential physiological and/or morphological characteristics) of NUN 70030 PPH selected from: 6) average stem length from cotyledons to first flower; 7) a concentrated fruit set; 8) average number of seeds per fruit; 9) average seed cavity length and 10) average leaf width.

Peppers according to the invention, such as the variety NUN 70030 PPH, or its progeny, or a plant having all physiological and/or morphological characteristics but one, two or three which are different from those of NUN 70030 PPH, can also be reproduced using vegetative reproduction methods. Therefore, the invention provides for a method of producing plants, or a part thereof, of variety NUN 70030 PPH, comprising vegetative propagation of variety NUN 70030 PPH. Vegetative propagation comprises regenerating a whole plant from a plant part of variety NUN 70030 PPH (or from its progeny or from or a plant having all physiological and/or morphological characteristics but one, two or three, which are different from those of NUN 70030 PPH), such as a cutting, a cell culture or a tissue culture.

The invention also concerns methods of vegetatively propagating a plant of the invention. In certain embodiments, the method comprises the steps of: (a) collecting tissue or cells capable of being propagated from a plant of the invention; (b) cultivating said tissue or cells to obtain proliferated shoots; and (c) rooting said proliferated shoots, to obtain rooted plantlets. Steps (b) and (c) may also be reversed, i.e., first cultivating said tissue to obtain roots and then cultivating the tissue to obtain shoots, thereby obtaining rooted plantlets. The rooted plantlets may then be further grown, to obtain plants. In one embodiment, the method further comprises step (d) growing plants from said rooted plantlets.

The invention also provides for a vegetatively propagated plant of variety NUN 70030 PPH (or from its progeny or from or a plant having all but one, two or three physiological and/or morphological characteristics which are different from those of NUN 70030 PPH, or a part thereof, having one or more distinguishing characteristics and/or all the morphological and physiological characteristics of NUN 70030 PPH (except for the characteristics differing), when grown under the same environmental conditions.

Parts of NUN 70030 PPH (or of its progeny or of a plant having all physiological and/or morphological characteristics but one, two or three which are different from those of NUN 70030 PPH) encompass any cells, tissues, organs obtainable from the seedlings or plants, such as but not limited to: pepper fruits or parts thereof, cuttings, hypocotyl, cotyledon, pollen and the like. Such parts can be stored and/or processed further. Encompassed are therefore also food or feed products comprising one or more of such parts, such as canned, chopped, cooked, roasted, in a salsa, frozen, dried, pickled, or powdered pepper fruit from NUN 70030 PPH or from progeny thereof, or from a derived variety, such as a plant having all but one, two or three physiological and/or morphological characteristics which are different from those of NUN 70030 PPH.

In one aspect haploid plants and/or double haploid plants of NUN 70030 PPH, or a plant having all but one, two or three physiological and/or morphological characteristics which are different from those of NUN 70030 PPH, or progeny of any of these, are encompassed herein. Haploid and double haploid (DH) plants can, for example, be produced by cell or tissue culture and chromosome doubling agents and regeneration into a whole plant. For DH production chromosome doubling may be induced using known methods, such as colchicine treatment or the like.

Also provided are plant parts derived from variety NUN 70030 PPH (or from its progeny or from a plant having all but one, two or three physiological and/or morphological characteristics which are different from those of NUN 70030 PPH D), or from a vegetatively propagated plant of NUN 70030 PPH (or from its progeny or from a plant having all but one, two or three physiological and/or morphological characteristics which are different from those of NUN 70030 PPH), being selected from the group consisting of: harvested fruits or parts thereof, pollen, cells, leaves or parts thereof, petioles, cotyledons, hypocotyls, shoots or parts thereof, stems or parts thereof, roots or parts thereof, cuttings, or flowers.

In one embodiment, the invention provides for extracts of a plant described herein and compositions comprising or consisting of such extracts. In a preferred embodiment, the extract consists of or comprises tissue of a plant described herein or is obtained from such tissue.

In still yet another aspect, the invention provides a method of determining the genotype of a plant of the invention comprising detecting in the genome (e.g., a sample of nucleic acids) of the plant at least a first polymorphism. The method may, in certain embodiments, comprise detecting a plurality of polymorphisms in the genome of the plant, for example by obtaining a sample of nucleic acid from a plant and detecting in said nucleic acids a plurality of polymorphisms. The method may further comprise storing the results of the step of detecting the plurality of polymorphisms on a computer readable medium.

The invention also provides for a food or feed product comprising or consisting of a plant part described herein wherein the plant part can be identified as a part of the plant of the invention. Preferably, the plant part is a pepper fruit or part thereof and/or an extract from a fruit or another plant part described herein. The food or feed product may be fresh or processed, e.g., dried, grinded, powdered, pickled, chopped, cooked, roasted, in a salsa, pickled, canned, steamed, boiled, fried, blanched and/or frozen, etc.

For example, containers such as cans, boxes, crates, bags, cartons, Modified Atmosphere Packagings, films (e.g., biodegradable films), etc. comprising plant parts of plants (fresh and/or processed) described herein are also provided herein.

Marketable pepper fruits are generally sorted by size and quality after harvest. Alternatively the pepper fruits can be sorted by pungency.

Peppers may also be grown for use in grafting or inosculation as rootstocks (stocks) or scions (cions). Typically, different types of peppers are grafted to enhance disease resistance, which is usually conferred by the rootstock, while retaining the horticultural qualities usually conferred by the scion. It is not uncommon for grafting to occur between cultivated pepper varieties and related Capsicum species. Methods of grafting and vegetative propagation are well-known in the art.

So in one aspect the invention relates to a plant comprising a rootstock or scion of NUN 70030 PPH.

Using methods known in the art like “reverse breeding”, it is possible to produce parental lines for a hybrid plant such as NUN 70030 PPH; where normally the hybrid is produced from the parental lines. Such methods are based on the segregation of individual alleles in the spores produced by a desired plant and/or in the progeny derived from the self-pollination of that desired plant, and on the subsequent identification of suitable progeny plants in one generation, or in a limited number of inbred cycles. Such a method is known from WO2014076249 or from Nature Protocols Volume: 9, Pages: 761-772 (2014) DOI: doi:10.1038/nprot.2014.049, which are enclosed by reference. Such method for producing parental lines for a hybrid organism, comprises the steps of: a) defining a set of genetic markers that are present in a heterozygous form (H) in a partially heterozygous starting organism; b) producing doubled haploid lines from spores of the starting organism: c) genetically characterizing the doubled haploid lines thus obtained for the said set of genetic markers to determine whether they are present in a first homozygous form (A) or in a second homozygous form (B); d) selecting at least one pair of doubled haploid lines that have complementary alleles for at least a subset of the genetic markers, wherein each member of the pair is suitable as a parental line for a hybrid organism.

Thus in one aspect, the invention relates to a method of producing a combination of parental lines of a plant of the invention (NUN 70030 PPH) comprising the step of making double haploid cells from haploid cells from the plant of the invention (NUN 70030 PPH) or a seed of that plant; and optionally crossing these parental lines to produce and collect seeds. In another aspect, the invention relates to a combination of parental lines produced by this method. In still another aspect said combination of parental lines can be used to produce a seed or plant of NUN 70030 PPH when these parental lines are crossed. In still another aspect, the invention relates to a combination of parental lines from which a seed or plant having all but one, two or three physiological and/or morphological characteristics which are different from those of NUN 70030 PPH can be produced or in another aspect, wherein a seed or plant having the distinguishing characteristics 1)-5) or 1)-10) of NUN 70030 PPH, as herein defined, can be produced when grown under the same environmental conditions. In still another aspect, the invention relates to a combination of parental lines from which a seed or plant having all the characteristics of NUN 70030 PPH as defined in Table 1 can be produced when grown under the same conditions.

All documents (e.g., patent publications) are herein incorporated by reference in their entirety.

### CITED REFERENCES

Acquaah, Principles of Plant Genetics and Breeding, 2007, Blackwell Publishing, ISBN-13: 978-1-4051-3646-4
ams.usda.gov/AMSv1.0/getfile?dDocName=STELDEV3003768
on the worldwide web at rhs.org.uk/Plants/RHS-Publications/RHS-colour-charts
on the worldwide web at upov.int/edocs/tgdocs/en/tg076.pdf
John Burt, Growing capsicums and chillies, Farmnote 64/99 ISSN 0726-934X
Ince et al., (2010) Biochem. Genet. 48:83-95
Kothari et al., (2010) Biotechnology Advances 28: 35-48
Thabuis et al., (2004) Theor Appl Genet 109:342-351)
Clewer, A. G., and D. H. Scarisbrick. 2001. Practical statistics and experimental design for plant and crop science. John Wiley & Sons Ltd., New York.
WO2014076249
Vos et al. 1995, Nucleic Acid Research 23: 4407-4414
Nature Protocols Volume: 9, Pages: 761-772 (2014) DOI: doi:10.1038/nprot.2014.049

### Examples

### Development of NUN 70030 PPH

The hybrid NUN 70030 PPH was developed from a male and female proprietary inbred line of Nunhems. The female and male parents were crossed to produce hybrid (F1) seeds of NUN 70030 PPH. The seeds of NUN 70030 PPH can be grown to produce hybrid plants and parts thereof (e.g., pepper fruit). The hybrid NUN 70030 PPH can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 70030 PPH is uniform and stable.

### DEPOSIT INFORMATION

A total of 2500 seeds of the hybrid variety NUN 70030 PPH were deposited according to the Budapest Treaty by Nunhems B.V. on ______, at or at the NCIMB Ltd., Ferguson Building, Craibstone Estate, Bucksburn, Aberdeen AB21 9YA, United Kingdom (NCIMB). The deposit has been assigned or NCIMB ______. A deposit of NUN 70030 PPH and of the male and female parent line is also maintained at Nunhems B.V. Access to the deposit will be available during the pendency of this application to persons determined by the Director of the U.S. Patent Office to be entitled thereto upon request. Subject to 37 C.F.R. §1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

“USDA descriptors” are the plant variety descriptors for pepper (Capsicum spp.)—Exhibit C of the U.S. Department of Agriculture, Agricultural Marketing Service, Science and Technology, Plant Variety Protection Office, Beltsville, Md. 20705, which can be downloaded from the world wide web at ams.usda.gov/AMSv1.0/getfile?dDocName=STELDEV3003768 and which is herein incorporated by reference in its entirety.

The most similar variety to NUN 70030 PPH is Piton or Piton a commercial variety from US Agriseeds. In Table 1 a comparison between NUN 70030 PPH and Piton is shown based on a trial in the USA. Trial location: Acampo Calif. USA, (coordinates: 38.192873° N, −121.232637° W). Transplanting date: Jul. 27, 2013, harvesting date Oct. 30, 2013.

Two replications of 50 plants each, from which 15 plants or plant parts were randomly selected to measure characteristics. In Table 1 the USDA descriptors of NUN 70030 PPH (this application) and reference Piton (commercial variety) are summarized.

TABLE 1

| | Application | |
|---|---|---|
| | variety | Comparison |
| | NUN 70030 | Variety |
| Physiological and/or morphological characteristics | PPH | “Piton” |
| 1. SPECIES: 1 = C. annuum 2 = C. frutescens | 1 | 1 |
| 3 = C. baccatum 4 = C. chinense 5 = Other (specify) | | |
| 2. MATURITY | | |
| (In Region of Best Adaptability): | | |
| Days from transplanting until mature green stage | 70 | 80 |
| Days from transplanting until mature red or yellow stage | 90 | 100 |
| Days from direct seeding until mature green stage | 105 | 115 |
| Days from direct seeding until mature red or yellow stage | 125 | 135 |
| 3. PLANT | | |
| Plant Habit: | 2 | 2 |
| 1= Compact 2 = Semi-spreading 3 = Spreading 4 = Other | | |
| Plant Attitude: | 1 | 1 |
| 1 = Erect 2 = Semi-erect 3 = Prostrate 4 = Other | | |
| Plant Height (cm) | 75.5 | 86.5 |
| Plant Width (cm) | 69.6 | 73.5 |
| Length of Stem from Cotyledons to First Flower (cm) | 24.9 | 21.9 |
| Length of Third Internode (from soil surface) (mm) | 23.3 | 12.7 |
| Basal Branches: | 2 | 1 |
| 1 = None 2 = Few (2-3) 3 = Many (more than 3) | | |
| Branch Flexibility: | 2 | 2 |
| 1 = Willowy (Cayenne Long Red) 2 = Rigid (Yolo Wonder L) | | |
| Stem Strength (Breakage Resistance): | 2 | 3 |
| 1 = Weak 2 = Intermediate 3 = Strong | | |
| 4. LEAVES: | | |
| Leaf Width (mm) | 47.3 | 62.5 |
| Leaf Length (mm) | 95.9 | 120.2 |
| Petiole Length (mm) | 51.8 | 55.6 |
| Mature Leaf Shape: 1 = Lanceolate 2 = Elliptic | 1 | 1 |
| Leaf Color: 1 = Light Green 2 = Medium Green 3 = Dark | 3 | 3 |
| Green 4 = Purple 5 = Other (specify) | | |
| Color Chart Name Code: RHS | Green N137D | Green 137B |
| Leaf and Stem Pubescence: 1 = Absent (Yolo Wonder L) | 1 | 1 |
| 2 = Light 3 = Moderate (Serrano) 4 = Heavy (Chili Piquin) | | |
| Margin Undulation: 1 = Absent 2 = V. Weak 3 = Weak | 2 | 2 |
| 4 = Medium 5 = Strong 6 = V. Strong | | |
| Blistering: 1 = Absent 2 = Very Weak 3 = Weak 4 = Medium | 1 | 2 |
| 5 = Strong 6 = Very Strong | | |
| 5. FLOWERS: | | |
| Number of Flowers per Leaf Axil | 1 | 1 |
| Number of Calyx Lobes | 5-6 | 5 |
| Number of Petals | 7 | 6 |
| Flower Diameter (mm) | 19.8 | 13.1 |
| Corolla Color: 1 = White 2 = Purple 3 = Other (Specify) | 1 | 1 |
| Corolla Throat Markings: 1 = Yellow (Tan) 2 = Purple | 1 | 1 |
| 3 = Other (Specify) | | |
| Anther Color: 1 = Yellow 2 = Purple 3 = Other (Specify) | 2 | 2 |
| Style Length: 1 = Less Than Stamen 2 = Same as Stamen | 1 | 2 |
| 3 = Exceeds Stamen | | |
| Self-Incompatibility: 1 = Absent 2 = Present | 1 | 1 |
| 6. FRUIT: | | |
| Group: | 7 | 7 |
| 1 = Bell (Yolo Wonder L) 2 = Pimiento (Pimiento | | |
| Perfection) 3 = Ancho (Mexican Chili) 4 = Anaheim Chili | | |
| (Sandia) 5 = Cayenne (Cayenne Long Red) 6 = Cuban | | |
| (Cubanelle) 7 = Jalapeno (Jalapeno) 8 = Small Hot | | |
| (Serrano) 9 = Cherry (Sweet Cherry) 10 = Short Wax | | |
| (Floral Gem) 11 = Long Wax (Sweet Banana) 12 = Tabasco | | |
| (Tabasco) 13 = Habanero (Scotch Bonnet) 14 = Other. | | |
| Immature Fruit Color: | 2 | 2 |
| 1 = Light Green (Cubanelle) 2 = Medium Green (Long Thin | | |
| Cayenne) 3 = Dark Green (Yolo Wonder L) 4 = Very Dark | | |
| Green (Ancho Chili) 5 = Yellow (Yellow Belle) 6 = Purple | | |
| (Violetta) 7 = Ivory (Twiggy) 8 = Other. | | |
| Immature Fruit Color: Color Chart Name Code: RHS | Green 137A | Green N137D |
| Mature Fruit Color: | 1 | 1 |
| 1 = Red (Yolo Wonder L) 2 = Orange 3 = Orange-Yellow | | |
| (Golden Calwonder) 4 = Brown (Mulatto) 5 = Ivory | | |
| 6 = Green (Permagreen) 7 = Salmon 8 = Lemon Yellow | | |
| 9 = Other. | | |
| Mature Fruit Color: Color Chart Name Code: RHS | Red 46A | Red-Orange N34A |
| Pungency: | 2 | 2 |
| 1 = Sweet (Yolo Wonder L) 2 = Hot (Jalapeno) | | |
| mg Capsaicin per gram dry fruit | NA | NA |
| Scoville Units (dry fruit) | 23,800 | 8,230 |
| Flavor: | 3 | 3 |
| 1 = Mild Pepper Flavor 2 = Moderate Pepper Flavor | | |
| 3 = Strong Pepper Flavor 4 = Other. | | |
| Fruit Glossiness: 1 = Dull 2 = Moderate 3 = Shiny | 2 | 2 |
| Surface Smoothness: 1 = Smooth (Yolo Wonder L) | 1 | 1 |
| 2 = Rough (Long Thin Cayenne) | | |
| Fruit Position: | 3 | 3 |
| 1 = Upright (Santaka) 2 = Horizontal 3 = Pendent (Jalapeno) | | |
| Calyx Shape: | 2 | 2 |
| 1 = Cup-shaped (Enveloping Fruit Base) 2 = Saucer-shaped | | |
| (Flat, Non-Enveloping) | | |
| Calyx Diameter (mm) | 24.9 | 24.7 |
| Fruit Length (mm) | 100.0 | 111.2 |
| Fruit Diameter at Calyx Attachment (mm) | 34.7 | 33.2 |
| Fruit Diameter at Mid-point (mm) | 38.2 | 36.0 |
| Flesh Thickness at Mid-point (mm) | 4.47 | 5.68 |
| Average Number of Fruits per Plant | 71.2 | 63.2 |
| Average Fruit Weight (gm) | 52.3 | 56.1 |
| Fruit Base Shape: | 2 | 2 |
| 1 = Cupped (Yolo Wond L), 2 = Rounded (Jalapeno) | | |
| Fruit Apex Shape: | 1 | 1 |
| 1 = Pointed (Long Thin Cayenne) 2 = Blunt (Yolo Wonder | | |
| L) | | |
| Fruit Shape: | 4 | 4 |
| 1 = Bell (Yolo Wonder L) 2 = Conical (Pimiento) | | |
| 3 = Elongate (Long Thin Cayenne) 4 = Oblong (Jalapeno) | | |
| 5 = Oblate (Sunnybrook) 6 = Globe (Red Cherry) | | |
| 7 = Other | | |
| Fruit Shape (Longitudinal Section, see attached pictures): | 6 | 6 |
| 1 = Flattened 2= Round 3 = Heart-shaped 4 = Square | | |
| 5 = Rectangular 6 = Trapezoid 7 = Narrow Triangular | | |
| 8 = Triangular 9 = Horn-shaped | | |
| Fruit Shape (Cross Section, at Level of Placenta): | 4 | 4 |
| 1 = Elliptic 2 = Triangular 3 = Quadrangular 4 = Circular | | |
| Fruit Set: 1 = Scattered 2 = Concentrated | 2 | 1 |
| Interloculary Grooves: | 1 | 1 |
| 1 = Absent 2 = Very Shallow 3 = Shallow 4 = Medium | | |
| 5 = Deep 6 = Very Deep | | |
| % Fruits with one locule | 0 | 0 |
| % Fruits with two locules | 0 | 0 |
| % Fruits with three locules | 20 | 25 |
| % Fruits with four locules | 80 | 75 |
| % Fruits with five or more locules | 0 | 0 |
| Average Number of Locules | 3.6 | 3.5 |
| Pedicel Length (mm) | 32.5 | 24.7 |
| Pedicel Thickness (mm) | 4.08 | 4.51 |
| Pedicel Shape: 1 = Straight 2 = Curved | 2 | 2 |
| Pedicel Cavity: 1 = Absent 2 = Present | 1 | 1 |
| 7 SEED: | | |
| Seed Cavity Length (mm) | 88.4 | 92.5 |
| Seed Cavity Diameter (mm) | 25.9 | 26.0 |
| Placenta Length (mm) | 43.7 | 48.8 |
| Number of Seeds per Fruit | 135.2 | 189 |
| Gm per 1000 seeds | 7.2 | 7.8 |
| Seed Color 1 = Yellow; 2 = Purple | 1 | 1 |
| 8. ANTHOCYANIN (1 = Absent; 2 = Weak; 3 = Moderate; | | |
| 4 = Strong): | | |
| Seedling Hypocotyl | 1 | 1 |
| Stem | 2 | 2 |
| Node | 2 | 2 |
| Leaf | 1 | 1 |
| Pedicel | 1 | 1 |
| Calyx | 1 | 1 |
| Fruit | 2 | 1 |
| DISEASE RESISTANCE (Rate from 1 (most susceptible) | | |
| to 9 (most resistant); leave blank if not tested; indicate | | |
| Race or Strain, when known) | | |
| A. Viruses: | | |
| Cucumber Mosaic Virus | 1 | 1 |
| Curly Top Virus | 1 | 1 |
| Pepper Mottle Virus | 1 | 1 |
| Potato Y Virus | 7 | 7 |
| Tobacco Etch Virus | 1 | 1 |
| Tobacco Mosaic Virus | 1 | 7 |
| B. Other Diseases and Insects: | | |
| Anthracnose (Gloeosporium piperatum) | 1 | 1 |
| Bacterial Spot (Xanthomonas vesicatoria) | 8 | 1 |
| Cercospora Leaf Spot (Cercospora capsici) | 5 | 1 |
| Nematode (Meloidogyne incognita acrita) | 1 | 1 |
| Phytophthora Root Rot (Phytophthora capsici) | 1 | 1 |
| Ripe Rot (Vermicularia capsici) | 1 | 1 |
| Southern Blight (Sclerotium rolfsii) | 1 | 1 |
| Verticillium Wilt (Verticillium dahliae) | 1 | 1 |

These are typical values. Values may vary due to environment. Other values that are substantially equivalent are also within the scope of the invention. N.A.=not applicable; n.r.=not recorded.

1. A plant, plant part or seed of pepper variety NUN 70030 PPH, wherein a representative sample of said seed has been deposited under Accession Number NCIMB ______.

2. A plant or part thereof grown from the seed of claim 1.

3. The plant part of claim 2, further defined as a leaf, pollen, an ovule, a fruit, a scion, a rootstock, cutting, flower or a part of any of these or a cell.

4. A capsicum plant, or a part thereof which does not significantly differ from the plant of claim 2 in any of the distinguishing characteristics selected from the group consisting of are 1) average number of fruits per plant; 2) heat of (dried) fruit expressed as Scoville units; 3) maturity (at region of adaptability), expressed as days from transplanting until mature red stage; 4) average fruit diameter at mid-point; 5) average fruit length; 6) average stem length from cotyledons to first flower; 7) a concentrated fruit set; 8) average number of seeds per fruit; 9) average seed cavity length and 10) average leaf width.

5. A tissue or cell culture of regenerable cells of the plant of claim 2.

6. The tissue or cell culture according to claim 5, comprising cells or protoplasts from a plant part selected from the group consisting of embryos, meristems, cotyledons, hypocotyl, pollen, leaves, anthers, roots, root tips, pistil, petiole, flower, fruit, seed, stem and stalks.

7. A pepper plant regenerated from the tissue or cell culture of claim 5, wherein the plant has all of the physiological and morphological characteristics of the plant of claim 2 as listed in Table 1 when determined at the 5% significance level.

8. A pepper plant regenerated from the tissue or cell culture of claim 5, wherein the plant has all of the physiological and morphological characteristics of the plant of claim 2 when determined at the 5% significance level

9. A method of producing of the plant of claim 2, or a part thereof, comprising vegetative propagation of the plant of claim 2.

10. The method of claim 9, wherein said vegetative propagation comprises regenerating a whole plant from a part of the plant of claim 2.

11. The method of claim 9, wherein said part is a cutting, a cell culture or a tissue culture.

12. A vegetative propagated plant of claim 2, or a part thereof, wherein the plant has all of the physiological and morphological characteristics of the plant of claim 2 when determined at the 5% significance level.

13. A method of producing a pepper plant, comprising crossing the plant of claim 2 with a second pepper plant one or more times, and selecting progeny from said crossing and optionally allowing the progeny to form seed.

14. A method of producing a pepper plant, comprising selfing the plant of claim 2 one or more times, and selecting progeny from said selfing, and optionally allowing the progeny to form seed.

15. Progeny of the plant of claim 2 obtained by further breeding with said variety.

16. The progeny of claim 14, wherein said progeny have all the distinguishing characteristics 1) to 5) or 1) to 10) of the pepper plant of claim 2 when grown under the same environmental conditions wherein the distinguishing characteristics are defined as 1) an average of 67.7 to 74.76 fruits per plant; 2) heat of (dried) fruit expressed as Scoville units of 22610 to 24990; 3) maturity (at region of adaptability), expressed as days from transplanting until mature red stage of 85.5 to 94.5; 4) an average fruit diameter at mid-point of 36.3 to 40.1 mm; 5) an average fruit length of 95.0 to 105.0 mm; 6) an average stem length from cotyledons to first flower of 23.7 to 26.1 cm; 7) a concentrated fruit set; 8) an average of 128.5 to 142.0 seeds per fruit; 9) an average seed cavity length of 84.0 to 92.8 mm and 10) an average leaf width of 44.9 to 49.7 mm.

17. The progeny of claim 15 comprising all the morphological and physiological characteristics of Table 1 when determined at the 5% significance level.

18. A pepper plant having one, two or three physiological and/or morphological characteristics which are different from those of the plant of claim 2 and which otherwise has all the physiological and morphological characteristics of the plant of claim 2 as listed in Table 1, when determined at the 5% significance level.

19. A food or feed product comprising the plant part of claim 3 wherein the plant part can be identified as a part of the plant of the invention.

20. A method of introducing a desired trait into a pepper plant comprising:
a) crossing a plant of claim 2 with a second pepper plant that comprises a desired trait to produce F1 progeny;
b) selecting an F1 progeny that comprises a desired trait;
c) optionally selfing the F1 progeny one or more times to produce F2, F3, or further generation selfing progeny,
d) crossing the selected F1 progeny or the selfing progeny with a plant of claim 2 to produce backcross progeny;
e) selecting backcross progeny comprising the desired trait and which otherwise has all or essentially all the physiological and morphological characteristic of the plant of claim 2; and optionally
f) repeating steps (d) and (e) one or more times in succession to produce selected higher backcross progeny that comprise the desired trait.

21. A pepper plant produced by the method of claim 19.

22. A pepper plant comprising at least a first set of the chromosomes of the plant of claim 2.

23. The plant of claim 2 further comprising a single locus conversion, wherein said plant has essentially all of the morphological and physiological characteristics of the plant of claim 2, optionally wherein the single locus conversion confers a trait selected from the group consisting of male sterility, herbicide tolerance, insect resistance, pest resistance, disease resistance, environmental stress tolerance, modified carbohydrate metabolism and modified protein metabolism.

24. A plant comprising the scion or rootstock of claim 3.

25. A method of producing a combination of parental lines of the plant of claim 2 comprising the step of making double haploid cells from haploid cells from the plant of claim 2 or a seed of claim 1.

26. A combination of parental lines produced by the method of claim 24.

27. The combination of parental lines of claim 25, from which a seed or plant having the distinguishing characteristics 1)-5) or 1)-10) or wherein the plant has all the physiological and morphological characteristics of the plant of claim 2 as listed in Table 1; when the characteristics are determined at the 5% significance level; 1) an average of 67.7 to 74.76 fruits per plant; 2) heat of (dried) fruit expressed as Scoville units of 22610 to 24990; 3) maturity (at region of adaptability), expressed as days from transplanting until mature red stage of 85.5 to 94.5; 4) an average fruit diameter at mid-point of 36.3 to 40.1 mm; 5) an average fruit length of 95.0 to 105.0 mm; 6) an average stem length from cotyledons to first flower of 23.7 to 26.1 cm; 7) a concentrated fruit set; 8) an average of 128.5 to 142.0 seeds per fruit; 9) an average seed cavity length of 84.0 to 92.8 mm and 10) an average leaf width of 44.9 to 49.7 mm.
